(19) United States
(12) Patent Application Publication
(10) Pub. No.: US 2026/0248842 A1
(43) Pub. Date: Aug. 27, 2026

(54) METHOD FOR TRANSIENT ACIDIFICATION OF WOUNDS

(71) Applicant: SePRO Corporation, Carmel, IN (US)

(72) Inventors: Freddie L. Singleton, Rogers, AR (US); Reid Henry Bowman, Rogers, AR (US); David Nicholas, Rogers, AR (US); Jeffrey Alan Keiser, Rogers, AR (US)

(73) Assignee: SePRO Corporation, Carmel, IN (US)

(21) Appl. No.: 19/160,081

(22) PCT Filed: Mar. 1, 2024
(86) PCT No.: PCT/US2024/018193

§ 371 Date: Aug. 27, 2025

Related U.S. Application Data
(60) Provisional application No. 63/488,162, filed on Mar. 2, 2023.
(60) Provisional application No. 63/544,137, filed on Oct. 13, 2023.
(60) Provisional application No. 63/544,140, filed on Oct. 13, 2023.

(51) Int. Cl. A61K 33/02; A61K 9/00; A61K 9/51; A61P 17/02; A61P 29/00

(52) U.S. Cl. CPC A61K 33/02; A61K 9/0014; A61K 9/51; A61P 17/02; A61P 29/00

(57) ABSTRACT

This disclosure provides the use of nanoparticles of self-assembled acidic molecular clusters (NP-AMCs) of ammonium salts to lower the pH of skin and underlying tissue of a human or other mammal as a means of stimulating the localized immune response to a wound.

### CROSS-REFERENCE TO RELATED APPLICATIONS

The present application claims the benefit of and priority to U.S. Provisional Application No. 63/488,162, filed Mar. 2, 2023; U.S. Provisional Application No. 63/544,137, filed Oct. 13, 2023; and U.S. Provisional Application No. 63/544,140, filed Oct. 13, 2023, each of which is hereby incorporated by reference in its entirety for all purposes.

### FIELD

The present disclosure relates to methods of treating a wound. The present methods enhance an immune response of skin and underlying tissue by acidifying the skin and underlying tissues with a topical application of a composition comprising nanoparticles of acidic molecular clusters of an ammonium salt.

### BACKGROUND

The skin of humans and other mammals has multiple functions that range from controlling moisture loss, serving as a protective barrier, regulating body temperature, and acting as a sensory organ. The skin also serves as an immune organ to detect infections and injuries, protects from ultraviolet radiation in sunlight, and produces vitamin D, among other functions.

Skin serves as a habitat for a wide variety of microorganisms (bacteria, fungi and viruses) that have immune functions, such as protecting against invading pathogens. Microorganisms that colonize and occupy ecological niches on the skin are collectively referred to as the skin's microbiome—members of the microbiome help prevent infections by excluding potentially pathogenic species. However, if the integrity of the skin is damaged, such as by trauma, or if the balance between the microbiome and pathogens is disrupted, such as by a medical condition, skin disease can occur.

A key property of skin is its pH. Published studies report epidermal pH values ranging from about 4.0 to about 7.0, depending on the location of the skin on the body. For example, a 2006 study reported that the natural skin of humans has a pH of 4.7, a value that favors the skin's natural microbiome and helps select against foreign species. Thus, the ability of the skin to minimize the likelihood of an infection occurring is the result of the physiological activities of the skin and those of commensal microorganisms that help maintain homeostasis of the surface of the skin.

For humans and other mammals the pH of tissues beneath the skin is regulated within species-specific ranges. Schwalfenberg (2012) summarized the pH of various organs of the human body (Schwalfenberg, G. K., 2012. The alkaline diet: is there evidence that an alkaline pH diet benefits health? Journal of environmental and public health, 2012). Skin and underlying tissue damaged by trauma have pH values outside the normal range for the particular location on the body. Furthermore, a decrease in the pH of mammalian tissue is a hallmark of the inflammatory process. Subsequently, during the healing process, the pH of a wound increases from acidic levels to alkaline levels.

The initial damage to the skin and/or underlying tissue caused by trauma or infections can occur by a variety of significantly different processes. After treatment of infections to inhibit the causative microorganism(s), repair of infected wounds and trauma-induced wounds generally follow the same path. As detailed below, the wound healing process allows a body to repair damaged skin and tissue.

Wound healing occurs in four stages that overlap. The stages are (1) hemostasis, (2) inflammation, (3) proliferation (also referred to as granulation and contraction) and (4) remodeling (also referred to as maturation). Hemostasis begins immediately after an injury. The initial response to an injury is vasoconstriction of damaged blood vessels and release of clotting factors to promote platelet aggregation in the wound to stop bleeding. When the blood clotting system is activated, fibrin clots form due to platelet activation. The fibrin clot is a matrix that blocks bleeding and acts as a scaffold for migrating inflammatory cells that respond to cytokines and chemokines released after an injury.

Tissue injury results in the release of an array of “signal” molecules, especially damage-associated molecular patterns (DAMPs) from damaged cells or, in the case of an infection, pathogen-associated molecular patterns (PAMPs). Macrophages and dendritic cells, among others, respond to DAMPs and/or PAMPs by initiating neutrophil recruitment. Neutrophils respond to the specific signal molecules, also referred to as chemo-attractants, by migrating to the wound. Neutrophils produce a wide range of receptors, including GPCRs, Fc receptors, adhesion receptors, cytokine receptors, and pattern recognition receptors which allow recognition and response to a wound. Neutrophils exhibit a multi-phase tactic to eliminate foreign substances and materials from a wound.

The inflammatory stage is highly complex and involves multiple aspects and processes. Immediately following an infection starting, injury, trauma, or other physical damage to skin and/or underlying tissue, the inflammatory process starts with activation of the innate immune system. Production and release of proinflammatory cytokines and chemokines begin in the early stages of the inflammatory process. Neutrophils and monocytes migrate to the wound site and the former begin phagocytosis of foreign materials and cellular debris in the injury site. Likewise, microbial cells and/or virus particles are phagocytized by neutrophils. As part of initial activities of neutrophils, antimicrobial substances such as reactive oxygen radicals (also referred to as “reactive oxygen species” [ROS]), cationic peptides, and the eicosanoids, prostaglandins and leukotrienes, are produced and released. In addition, proteases and myeloperoxidase, an enzyme that produces hypochlorite anion using chlorine and hydrogen peroxide as substrates, are produced and contribute to the inactivation of microbial cells and virus particles. Devitalized tissue is removed by the action of matrix metalloproteinases (MMPs). Monocytes that were recruited to the wound differentiate into macrophages which then infiltrate the wound and phagocytize the neutrophils that have undergone apoptosis. Macrophages help facilitate the transition from inflammation to the proliferation stage.

When the inflammatory stage begins to decline, usually after about 2 to 5 days, the proliferation stage begins and also involves multiple processes. Endothelial cells proliferate, migrate and branch to form new blood vessels. Endothelial cells also produce a range of pro-inflammatory and anti-inflammatory cytokines and chemokines (compounds that attract certain types of cells) as well as growth factors. The primary outcome of the proliferation stage is to generate a cellular covering of the wound surface (i.e., re-epithelialization), restore the vascular network (i.e., vessels and capillaries), and form granulation tissue.

As the proliferation stage progresses, the remodeling phase begins. This phase of the healing process is designed to maximize tensile strength and restore the morphology and function of tissue. The remodeling phase is essentially coupled with the inflammatory process. As inflammation decreases, cellular proliferation increases, and three interconnected processes occur: (1) re-epithelialization; (2) restoring the vascular network; and (3) formation of granulation tissue. After granulation tissue has formed by activated fibroblasts, the remodeling phase starts. During remodeling, granulation tissue is eventually replaced by normal connective tissue and circulation is re-established. Once the remodeling process is completed, the remodeled tissue has fewer blood vessels and, concomitantly, less blood flow. Remodeled skin will be devoid of hair follicles and sebaceous glands. Also, the skin has reduced tensile strength. For example, healed skin will have up to about 20% less tensile strength than the original, undamaged skin. It will require about 6 to 24 months for collagen to mature.

One of the common aspects of wound healing is that the quality and rate of healing are affected by several factors such overall health of a human or other mammal, age and gender, presence or absence of infectious microorganisms, including viruses, and presence of other diseases, among others. There is no universally applicable treatment for different types of wounds. For example, depending on the nature of a wound, treatment can range from letting the body resolve and heal the wound to using medicaments to treat or prevent infections and increase the rate of healing.

Although much progress has been made in preventing and treating wound infections, there remains a need for more effective, less expensive, and straight-forward treatment methods and protocols that can be applied to a wide range of type of wounds (e.g., acute and chronic wounds) and other maladies of the skin and underlying tissues of humans and other mammals. The present invention satisfies these and other needs.

### SUMMARY

Disclosed herein are methods and compositions for treating wounds by acidifying the wound to promote healing. The methods involve applying to an area of skin including the wound a composition that includes nanoparticles that are self-assembled acidic molecular complexes (“NP-AMCs”) in water, or optionally in combination with one or more pharmaceutically acceptable excipients or carriers. The NP-AMCs permeate into and through the wound bed and underlying tissue to transiently acidify the environs of the wound to stimulate a local immune response. As detailed herein, this invention provides a novel method and use of NP-AMCs to selectively impart transient acidification of a wound of the skin and underlying tissue, especially during the early stages of wound healing. The method and use of NP-AMCs provides a means of enhancing the immune response to a wound to increase the rate of healing as well as a treatment for chronic wounds.

Disclosed herein are methods for treating a damaged area of skin having a wound or a skin disorder, the method comprising applying to the area of skin a composition comprising a pharmaceutically acceptable carrier and a plurality of nanoparticles, each nanoparticle comprising a self-assembled acidic molecular complex comprising an ammonium salt and an acid (“NP-AMC”). The composition is applied in an amount effective to reduce a pH in an area of the damaged are of skin to produce a treated area of skin.

In some embodiments, the pH of the treated area of skin is from pH 2 to pH 7, or from pH 3 to pH 6, or from pH 3 to pH 5. Optionally, the pH of the treated area of skin is lower than the pH of the damaged area of skin by 1 pH unit, 2 pH units, 3 pH units, 4 pH units, or 5 pH units. Optionally, the pH of the treated area of skin remains lower than the H of the damaged area of skin for a time period ranging from about 5 minutes to about 6 hours, or from about 10 minutes to about 4 hours, or from about 30 minutes to about 2 hours.

In some embodiments, the concentration of the NP-AMC in the composition is from about 0.005% to about 50% by weight, or from about 0.01% to about 10% by weight, or from about 0.1% to about 5% by weight. In some embodiments, the NP-AMC have an average diameter ranging from about 5 nm to about 2000 nm, or from about 10 nm to about 1000 nm, or from about 100 nm to about 400 nm. Optionally, the pH of the area of the skin is reduced to a pH ranging from pH 2 to pH 7, or from pH 3 to pH 6, or from pH 3 to pH 5.

In some embodiments, the composition is in the form of a solution, a suspension, an emulsion, a powder, a gel, an ointment, a cream, or a paste. Optionally, the pH of the composition ranges from a pH of 1 to a pH of 6, or from a pH of 3 to a pH of 5.

In some embodiments, the skin is human skin. In alternative embodiments, the skin is animal skin. Optionally, the wound or the skin disorder comprises a trauma site or a site of infection.

In some embodiments, applying the composition comprises topically applying the composition to the damaged area of skin and to skin and/or tissue surrounding the damaged area of skin. In some embodiments, the wound is selected from the group consisting of a scrape, a scratch, a cut, an abrasion, a puncture, an animal or insect bite or sting, and a reaction to contact with a chemical that damages healthy skin. Optionally, the wound is an acute wound. Alternatively, the wound is a chronic wound.

In some embodiments, the ammonium salt is selected from the group consisting of ammonium chloride (NH₄Cl), ammonium sulfate ((NH₄)₂SO₄), ammonium bisulfate (NH₄HSO₄), ammonium carbonate ((NH₄)₂CO₃), ammonium bicarbonate (NH₄HCO₃), ammonium iron(II) sulfate ((NH₄)₂Fe(SO₄)₂.6H₂O), ammonium formate (NH₄HCO₂), ammonium acetate (C₂H₇NO₂), ammonium borate (H₁₂BN₃O₃), ammonium nitrate (NH₄NO₃), and ammonium phosphate ((NH₄)₃PO₄). In some embodiments, the acid is selected from the group consisting of acetic acid, boric acid, carbonic acid, hydrochloric acid, nitric acid, phosphoric acid, sulfamic acid, sulfuric acid, and formic acid.

In some embodiments, the ammonium salt is selected from the group consisting of ammonium chloride (NH₄Cl), ammonium carbonate ((NH₄)₂CO₃), ammonium bicarbonate (NH₄HCO₃), ammonium iron(II) sulfate ((NH₄)₂Fe(SO₄)₂.6H₂O), ammonium formate (NH₄HCO₂), ammonium acetate (C₂H₇NO₂), ammonium borate (H₁₂BN₃O₃), ammonium nitrate (NH₄NO₃), and ammonium phosphate ((NH₄)₃PO₄). In some embodiments, the acid is selected from the group consisting of acetic acid, boric acid, carbonic acid, hydrochloric acid, nitric acid, phosphoric acid, sulfamic acid, and formic acid.

In some embodiments, the ammonium salt is ammonium sulfate ((NH₄)₂SO₄) and/or ammonium bisulfate (NH₄HSO₄) and the acid is sulfuric acid. In some embodiments, the NP-AMCs comprises sulfuric acid, ammonium sulfate, and water, and wherein, once applied to the area of skin, the NP-AMCs dissociates to ionic chemical species comprising NH₄⁺, SO₄²⁻, and H₃O⁺ ions.

In some embodiments, the wound or skin disorder comprises an infectious microorganism, and the composition is applied in an amount effective to inhibit the growth of the infectious microorganism.

In some embodiments, the treating comprises reducing inflammation. In some embodiments, the composition is applied in an amount effective to stimulate a signaling pathway to downregulate the production of a proinflammatory immune factor. Optionally, the proinflammatory immune factor(s) is a proinflammatory cytokine selected from the group consisting of IL-1β, IL-6, IL-8, and TNF-α.

Optionally, in any method described herein, the composition does not include a metal.

### BRIEF DESCRIPTION OF THE DRAWINGS

FIG. 1 shows a schematic of NP-AMCs applied to skin at an initial time. Small gray spheres=water molecules, large black spheres=NP-AMCs. ABS=ammonium bisulfate, AMS=ammonium sulfate, H⁺=proton.

FIG. 2 shows a schematic of the mode-of-action of NP-AMCs after application to skin. Small gray spheres=water molecules, large black spheres=NP-AMCs. ABS=ammonium bisulfate, AMS=ammonium sulfate, H⁺=proton.

FIG. 3. shows a bar graph of the average CPEK cell viability percent (LDH assay) for CPEK before and after exposure to NP-AMCs at different concentrations, house dust mites, and a combination of both for 24 hours.

FIG. 4. shows a bar graph of the average CPEK cell viability as a function of luminescence (CellTiter-Glo® 2.0 assay) for CPEK before and after exposure to NP-AMCs, house dust mites, and a combination of both for 24 hours.

FIG. 5. shows a bar graph of the median CPEK inflammatory cytokine production (Interleukin-8 [IL-8]) before and after exposure to NP-AMCs, house dust mites, and a combination of both for 24 hours.

FIG. 6. shows a bar graph of the median CPEK inflammatory cytokine production (Keratinocyte-derived chemokine [KC]) before and after exposure to NP-AMCs, house dust mites, and a combination of both for 24 hours.

FIG. 7. Shows a photograph of pig skin samples with locations of formulations containing NP-AMCs.

FIG. 8. is a photograph of a paper towel saturated with pH indicator solution after a 6-hour incubation period under pig skin samples containing NP-AMCs. A color change from blue green to pink illustrates a pH of 3-4 pH units.

FIG. 9. Shows three photographs of a pig skin sample treated with a solution of NP-AMCs on Whatman #3 filter paper saturated with pH indicator solution. Panel A is a photograph taken at time=0 (immediately after placing the 10 μL samples at the locations indicated by the dots. Panels B and C are photographs of the pig skin top and bottom, respectively, taken after 4 hr incubation.

FIG. 10. Shows photographs taken initially and after 60 minutes, of full thickness wounds in porcine skin treated with 100 μg of DermaBase Cream amended with 2.5% (w/w) of a 20% (w/w) NP-AMCs solution.

FIG. 11. Shows two photographs comparing wound treatments in an untreated control, a wound treated with Anthocyanin pH indicator, and a wound treated with DermaBase Cream amended with 2.5% (w/w) of a 20% (w/w) NP-AMCs solution. Photograph A was taken after addition of anthocyanin pH indicator. Photograph B was taken after an additional 30 min incubation time.

FIG. 12. Shows three photographs. Photograph A was taken at time=0, immediately after treatment of skin with two volumes of 10% NP-AMCs solution. Photograph B was taken after the treatment and after 18 hrs of incubation at room temperature. Photograph C was taken after the skin sample was removed and the Petri dish with the absorbent paper layer was placed over a LED light.

FIG. 13. Shows images of the hand of a 69-year-old male Caucasian with a cat bite wound treated with cream containing NP-AMCs over a period of six days.

### DETAILED DESCRIPTION

### A. Definitions

Unless defined otherwise, all technical and scientific terms used herein have the same meaning as is commonly understood by one of ordinary skill in the art to which this invention pertains. Although methods and materials similar or equivalent to those described herein can be used in the practice of the present invention, suitable methods and materials are described below. If a definition set forth in this section is contrary to or otherwise inconsistent with a definition set forth in a patent, application, published application, or other publication that is herein incorporated by reference, the definition set forth in this section prevails over the definition that is incorporated herein by reference.

This specification includes references to “one embodiment” or “an embodiment.” The appearances of the phrases “in one embodiment” or “in an embodiment” do not necessarily refer to the same embodiment, although embodiments that include any combination of the features are generally contemplated, unless expressly disclaimed herein. Particular features, structures, or characteristics may be combined in any suitable manner consistent with this disclosure.

As used herein, the term “transient acidification” refers to a temporary or short-lived increase in the number of protons sufficient to cause a decrease in the pH of a local environment, for example, in the skin and/or the underlying tissue.

The terms “molecular complex” and “molecular cluster” refer to a homogeneous or heterogenous aggregate, cluster, or group of molecules and elements that self-assemble and have a specific composition and arrangement.

As used herein, “tissue” refers to the various groups of cells that make up organs and other body parts. It is well known and understood that the bodies of humans and other animals have bodies composed of well-organized tissues that are grouped according to cell type and function. These include muscle, epithelial, connective, and nervous tissue.

As used herein, “pharmacologically active,” “biologically active” and “active” when used to describe a compound, substance, or material refer to a property of the compound, substance, or material of having an effect or influence on the physiology or a biological function or process of a cell, tissue, or organism. Thus, a biologically active compound, substance, or material may have a beneficial or detrimental effect on a cell, tissue, or organism, depending on the desired outcome. For example, “pharmacologically active,” “biologically active,” or “active” compounds, substances, or materials can be used to treat a disease and/or symptom or a disease, including pain. One skilled in the art will understand that such compounds are used in different manners, depending on the disease or malady. For example, such compounds can be ingested, injected, or applied to the skin as a solution, suspension, lotion, emulsion, gel, ointment, cream, or paste.

As used herein, the terms “pharmacologically acceptable excipients,” “dermatologically acceptable excipients,” and “inactive ingredients” refer to compounds, substances, and materials having no biological activity that are formulated with biologically active ingredients to impart desired characteristics on the final product. For example, excipients that may be present in the disclosed compositions include, but are not limited to, emulsifiers, penetration enhancers, absorption promoters, antioxidants, metal chelators, humectants, and fragrances that act as carriers of active substances, provide even spreading of a formulation, etc.

As used herein, “local immune response” refers to the cell types, cellular processes, and chemical and biochemical substances produced by the body in the environs of any manner of physical or chemical damage to the skin or other body tissue.

As used herein, “inflammatory process” refers to the body's natural response to trauma, injury or other physical damage that cause cellular damage and/or introduction of materials or substances foreign to the body. As used herein, “foreign materials” and “foreign substances” refer to any substance, material, or chemical substance not produced by the body as well as any virus, bacteria, fungus, or other type of microorganism or macroorganism.

As used herein, “topically applied” or “topical application” refers to the placement of a substance, material, or formulation to the outermost layer of the skin of a human or animal.

The term “about,” as used herein when referring to a measurable value such as an amount or concentration and the like, is meant to encompass variations of within 10% of the specified amount.

As used herein, “skin” refers to the thin layer of tissue forming the natural outer covering of the body of a human or other animal and includes all cell types and sublayers of the epidermis and the dermis.

As used herein, “wound” refers to an injury to the skin and underlying tissue caused by a cut, abrasion, impact, or contact with an object or material. “Wound” further refers to cuts and scrapes known as open wounds, as well as others, such as deep bruises, or closed wounds. Non limiting examples of wounds suitable for treatment in accordance with the present disclosure include abrasions such as those caused by scraping to the outer layer of skin; incisions such as those caused by sharp edges, knives, metal edges, broken glass or other sharp object; lacerations or jagged, irregular cuts or tears of the skin; punctures such as those caused by an object piercing the skin layers and creating a small hole; and/or burns. Additional non-limiting wounds suitable for treatment in accordance with the present disclosure include puncture wounds, gaping wounds, wounds having fatty layers, tissue or muscle exposed, wounds having one or more foreign bodies therein, wounds causing severe pain, wounds having blood flowing therefrom, or any wound that causes numbness or loss of movement below the wound. The term “wound” also includes damage to the skin and underlying tissue by contact with an object or material at a high temperature or very low temperature so that damage to cells and tissue occurs. Likewise, contact with certain chemicals that damage the skin and/or underlying tissues cause wounds. In the context of this disclosure, sustained contact with an object or material includes that experienced by a body confined to a bed, wheelchair, or other situation in which prolonged contact results in injuries to the skin and underlying tissues because of prolonged pressure on the skin. Such wounds are referred to as pressure ulcers or bedsores. Certain diseases or dysfunctional states of the body such as diabetes cause wounds.

As used herein, “nanoparticle” refers to a molecular assemblage comprised of one or more acidic molecular clusters that self-assemble into a three-dimensional geometric shape with a diameter in the range of about 5 nm to about 2,000 nm, preferably in the range of about 50 nm to 500 nm. As used herein, the term nanoparticle includes, but is not limited to a NP-AMC which is a self-assembling molecular cluster of an ammonium salt, an acid, and water.

### B. Overview of the Invention and Embodiments

Methods and compositions disclosed herein treat wounds using transient, i.e., temporary, acidification to promote healing. The methods involve applying to wounded or damaged skin a composition that includes nanoparticles that are self-assembled acidic molecular complexes (“NP-AMC”) in combination with a pharmacologically acceptable excipient or carrier.

The compositions disclosed herein include a plurality of nanoparticles, where each nanoparticle (“NP-AMC”) is a self-assembled molecular complex comprising an ammonium salt and an acid. The NP-AMCs self-assemble in an aqueous matrix that maintains the molecular complex in hydrated form and also acts as a carrier for the NP-AMCs in the absence of any other carrier or excipient. The NP-AMCs are indefinitely stable in an aqueous medium and in pharmacologically acceptable formulations that include water as an ingredient. The NP-AMCs in the compositions disclosed herein are three-dimensional molecular complexes stabilized by ionic attractive forces and hydrogen bonding. The NP-AMC are produced by mixing an amine solution and an acid-containing solution under conditions that facilitate in situ formation, i.e., self-assembly, of the acidic molecular clusters.

During early studies on the NP-AMCs it was discovered that NP-AMCs prepared with sulfuric acid and ammonia permeated pig skin samples and caused transient acidification of the skin and underlying tissue. When solutions of ammonium sulfate and ammonium bisulfate were prepared without NP-AMCs but with the same amounts of ammonia equivalents and mixed with the same amount of sulfuric which resulted in the formation of NP-AMCs as described in U.S. Pat. No. 9,938,171, neither of the acidified ammonium salts solutions permeated pig skin and caused transient acidification.

Optionally, the compositions disclosed herein include a pharmacologically acceptable excipient, such as to confer a desired property on the composition. Optionally, the compositions disclosed herein can be combined with a pharmacologically acceptable carrier or device, such as a cream, hydrogel, or bandage, to facilitate application of the NP-AMCs.

The ammonium salt of the NP-AMC can include one or more of ammonium chloride (NH₄Cl), ammonium sulfate ((NH₄)₂SO₄), ammonium bisulfate (NH₄HSO₄), ammonium carbonate ((NH₄)₂CO₃), ammonium bicarbonate (NH₄HCO₃), ammonium formate (NH₄HCO₂), ammonium acetate (C₂H₇NO₂), ammonium borate (H₁₂BN₃O₃), ammonium nitrate (NH₄NO₃), and ammonium phosphate ((NH₄)₃PO₄). The acid of the NP-AMC can include one or more of acetic acid, boric acid, carbonic acid, hydrochloric acid, nitric acid, phosphoric acid, sulfamic acid, sulfuric acid, and formic acid.

The ammonium salt of the NP-AMC can include one or more of ammonium chloride (NH₄Cl), ammonium carbonate ((NH₄)₂CO₃), ammonium bicarbonate (NH₄HCO₃), ammonium formate (NH₄HCO₂), ammonium acetate (C₂H₇NO₂), ammonium borate (H₁₂BN₃O₃), ammonium nitrate (NH₄NO₃), and ammonium phosphate ((NH₄)₃PO₄). The acid of the NP-AMC can include one or more of acetic acid, boric acid, carbonic acid, hydrochloric acid, nitric acid, phosphoric acid, sulfamic acid, and formic acid.

In some examples, the NP-AMCs include molecules of each of ammonium sulfate, ammonium bisulfate, sulfuric acid, and optionally water. Those NP-AMCs have an empirical formula of

((NH₄)₂SO₄)ₐ.(H₂SO₄)_{b}.(H₂O)_{c}.(NH₄HSO₄)ₓ (I)

where a is between 1 and 5, b is between 1 and 5, c is between 0 and 5, and x is between 1 and 20. Those NP-AMCs can be formed by combining streams of water, concentrated sulfuric acid, and anhydrous liquid ammonia according to methods disclosed by Nicholas et al. in U.S. Pat. No. 9,938,171, which is incorporated herein by reference in its entirety.

In other examples, other acid and amine sources can be used in the production process to form NP-AMCs that include molecules of other ammonium salts and/or other acids. Methods of producing NP-AMCs using amines and acids other than ammonia and sulfuric acid are disclosed in U.S. Pat. Nos. 10,807,889 and 10,544,055 and U.S. Patent Application Publication 2022/0024865, the teachings of each of which are incorporated herein by reference in their entireties.

The average diameter of the NP-AMCs is generally in the range of about 5 nm to about 2,000 nm, such as from about 10 nm to about 1000 nm or from about 100 nm to about 400 nm. In some examples, however, the size can be less than about 10 nm or more than about 2,000 nm. The size of the NP-AMCs depends in part on the starting materials and production process parameters used. For example, both the type of acid and the amine source affect the size of the NP-AMC. Also, the temperature, pressure, and other process parameters used during production can affect the size of the NP-AMC.

A composition for use in the methods disclosed herein can include NP-AMCs in water with no additional ingredients. Alternatively, the composition can further include one or more pharmaceutically acceptable excipients or carriers. The compositions can be prepared by any of the methods well known in the art of pharmacy and drug delivery. In general, methods of preparing the compositions include the step of bringing the NP-AMCs into association with a carrier optionally containing one or more accessory ingredients. The compositions are typically prepared by uniformly and intimately bringing the NP-AMCs as the active ingredient into association with a liquid carrier or an emulsion base or both, and then, if necessary, shaping the product into the desired formulation (e.g., cream, lotion, or gel) having the desired viscosity. The compositions can be conveniently prepared and/or packaged in unit dosage form.

In some examples, the methods disclosed herein may be carried out by topically administering the composition to the site of the wound, e.g., topically applying the composition to the site of the wound. This topical administration can be carried out by any suitable means. In one aspect, a topical composition comprising NP-AMCs is in the dosage form of a gel, an emulsion (including lotion, cream, and milk), a foam, a suspension, a liquid, a spray, a paste, or an ointment. In some examples, the composition is applied to the skin surface using a finger or an applicator. In other examples, the composition is disposed on a bandage, which is applied to the site of the wound. In certain examples, the topical composition comprising NP-AMCs is embedded in the non-adhesive absorbent pad of a bandage. In certain examples, the topical composition is an oil-in-water emulsion, in which an internal oil phase is dispersed in a continuous aqueous phase. Alternatively, water-in-oil-in-water, water-in-oil, and oil-in-water-in-oil emulsions are also contemplated. The emulsion may be a macroemulsion, a microemulsion, or a nanoemulsion.

In addition to the NP-AMCs, the compositions comprise one or more pharmaceutically acceptable excipients, especially one or more dermatologically acceptable excipients, such as liquid oils, waxes, viscosity-modifying agents, thickening agents, gelling agents, alcohols, surfactants, chelating agents, buffers, preservatives, humectants, emollients, stabilizers, diluents, dispersing agents, emulsifiers, wetting agents, stabilizers, pH adjusters, solvents or cosolvents.

In a one embodiment, the composition is an oil-in-water emulsion comprising an oil phase and an aqueous phase, an emulsifier, and one or more of the pharmaceutically or dermatologically acceptable excipients as described above. In this embodiment, the oily phase can be a vegetable oil, for example olive oil or arachis oil, or a mineral oil, for example liquid paraffin or mixtures of these. Suitable emulsifying agents can be naturally occurring gums, such as gum acacia or gum tragacanth; naturally occurring phospholipids, such as soy lecithin; esters or partial esters derived from fatty acids and hexitol anhydrides, such as sorbitan monooleate; and condensation products of said partial esters with ethylene oxide, such as polyoxyethylene sorbitan monooleate. Other oils and emulsifying agents suitable for use in the compositions or formulations of the present invention will be known to and used by those of skill in the art.

In some embodiments, the emulsion for the NP-AMCs is a commercially available emulsion, wherein the NP-AMCs are added to or mixed with the commercially available emulsion. Suitable commercially available emulsions for use in the compositions disclosed herein include, DermaBase Cream (NDC 0574-0071-16), which is an oil-in-water emulsion base that is commercially available from Padlock Laboratories or Macelle. Any suitable emulsion base, which is often referred to as a “DermaBase” by those in the art, can be used in combination with NP-AMCs to form the compositions or formulations of the present invention.

The effects described herein can be achieved using NP-AMCs as the only active ingredient, so it is not necessary to include other biologically active agents in the compositions. Accordingly, in some examples, the compositions do not include a biologically active ingredient other than the NP-AMC. In particular, in some examples, compositions disclosed herein do not include a metal. More specifically, in some examples, the compositions do not include an antimicrobial metal. In other examples, however, additional biologically active agents may be included in the compositions and may enhance certain effects or provide additional benefits. In those examples, antibiotics such as clarithromycin, amoxicillin, and metronidazole should be stable in diluted solutions of PABS.

Compositions described herein can be in any form suitable for topical application to skin. In some examples, the compositions are in the form of form of a solution, a suspension, an emulsion, a powder, a gel, an ointment, a cream, or a paste.

The compositions described herein include NP-AMCs in a concentration range from about 0.001% to about 50%, such as from about 0.005% to about 50%, from about 0.01% to about 10%, or from about 0.1% to about 5%, all percentages by weight.

In some examples, a composition to be topically applied to skin is a water-in-oil emulsion or an oil-in-water emulsion and includes NP-AMCs in a concentration range from about 0.001% to about 5% by weight. In some examples, the NP-AMCs are present in the composition in a concentration range from about 0.01% to about 15%, and the NP-AMCs include about 2% to about 5% sulfuric acid and about 5% to about 7% water, all percentages by weight.

The NP-AMCs are acidic, and the compositions disclosed herein have a pH ranging from a pH of 1 to a pH of 6, such as a pH of 3 to a pH of 5, and including a pH of about 1, about 2, about 3, about 4, about 5, or about 6.

It has been surprisingly discovered that formulations containing NP-AMCs can be applied to any manner of wounds or infected sites of the skin to acidify the skin and underlying tissues. The pH of healthy muscle tissue in the human body is slightly acidic and ranges from 6.5 to 7.5. A wound tends to be slightly acidic in the early stage and becomes alkaline as healing progresses. Notably, different healthy organs/tissues have different pHs. Acidification of a wound stimulates the local immune response and healing process. In some examples, controlled acidification can be based on the concentration of NP-AMCs in the formulation, frequency of application, and the amount applied to a surface (usually about ~1 mm think layer). The methods and compositions are suitable for use on the skin of humans and other animals, including but not limited to household pets, such as cats and dogs; livestock, such as sheep, cattle, and horses; and other small and large animals.

The methods and compositions are suitable for use on any type of wound, damage to skin, or skin disorder (collectively “wounds”), including closed wounds where the skin remains intact or open wounds where the skin is broken. A wound can be the result of any injury to the skin and underlying tissue, such as damage caused by impact or contact with an object or material. In some examples, the wound can be a trauma site or a site of infection. For example, the wound can be a scrape, a scratch, a cut, an abrasion, an incision, a laceration, a puncture, or an animal or insect bite or sting. The wound or skin disorder can be caused by contact with a chemical that damages healthy skin. The wound can be the result of sustained contact with an object or material, including that experienced by a body confined to a bed, wheelchair, or other situation in which prolonged contact results in injuries to the skin and underlying tissues because of prolonged pressure on the skin. Such wounds are referred to as pressure ulcers or bedsores. The wound can be caused by a disease or dysfunctional state of the body, such as diabetes. The methods and compositions described herein are suitable for use on any and all of the foregoing wounds.

The methods and compositions described herein are suitable for use on acute wounds and chronic wounds. The initial damage to the skin and/or underlying tissue caused by trauma or infections is considered an acute wound. Immediately after a wound occurs, the body responds by initiating the healing process. Most wounds progress through the different stages of healing. In some instances, however, a wound does not heal, begins to heal but healing progresses very slowly, or fails to progress through all healing stages; such wounds are referred to as “chronic wounds.” Chronic wounds are characterized by pathological processes that require appropriate medical intervention. For example, a chronic wound may have continuous inflammation, persistent and difficult to treat infections, and tissue necrosis, among others. As detailed herein, both acute and chronic wounds can be treated with the compositions and methods described herein.

In methods described herein, a composition including NP-AMCs is topically applied to an area of skin having a wound or skin disorder. For such topical application, the compositions can be in any form suitable for topical application to skin, such as but not limited to a solution, a suspension, an emulsion, a powder, a gel, an ointment, a cream, or a paste. Methods of application include topical methods, including spraying, as drops, in droplets generated with a nebulizer, steamer, or vaporizer. While topical application is the most convenient means of treating a wound with the disclosed compositions, those skilled in the art will recognize that other methods of application are possible.

The composition penetrates the skin and enters the underlying tissue, including the wound bed or affected area of the skin. The composition is applied in an amount sufficient to reduce the pH of the skin, underlying tissue, wound, and/or affected area to cause transient (temporary) acidification of the damaged skin and underlying tissue stimulate a local immune response and facilitate healing.

Applying a composition including NP-AMCs to skin lowers the pH of the area of skin and underlying tissue, acidifying the wound and surrounding tissue for a period of time. In some examples, applying the composition reduces the pH of the area of skin and underlying tissue by about 0.5 pH unit to more than about 5 pH units. In some examples, applying the composition reduces the pH by about 1 pH unit, 2 pH units, 3 pH units, 4 pH units, or 5 pH units. In some examples, applying the composition to skin reduces the pH to a value ranging from pH 2 to pH 7, such as from pH 4 to pH 6, from pH 3 to pH 6, or from pH 3 to pH 5, or from pH 3.5 to pH 5.

Acidification of the skin and underlying tissues by the NP-AMCs is temporary due to the body's natural buffering, and thus the acidification is referred to herein as transient acidification. As used herein, the term “transient acidification” refers to a temporary or short-lived increase in the number of protons sufficient to cause a decrease in the pH of a local environment, for example, in the skin and/or the underlying tissue. Such transient acidification follows application of a composition containing NP-AMCs. After NP-AMCs are applied and reduce the pH of tissue, buffering by bodily fluids restores the natural pH. Although temporary, such transient acidification of damaged skin and underlying tissue stimulates the local immune response and promotes the body's inherent healing process.

In some examples, one application of a composition including NP-AMCs reduces the pH of an area of skin and underlying tissue, acidifying the wound and tissue for a time period ranging from about 1 minute to about 8 hours, such as from about 5 minutes to about 6 hours, from about 10 minutes to about 4 hours, from about 1 hour to about 6 hours, from about 1 hour to about 4 hours, or from about 30 minutes to about 2 hours. Subsequent applications of the composition to the same area of skin increase the time period during which the pH is reduced.

In some examples, after NP-AMCs are applied and reduce the pH of tissue to a value in the range of about 2 to about 7, buffering by bodily fluids restores the natural pH of the tissue to a value in the range of about 7.35 to about 7.45 within a time range of about 1 minute to about 8 hours, or in a time range of about 1 hour to about 6 hours. In some examples, after NP-AMCs are applied and reduce the pH of tissue to a value in the range about 4 to about 6, buffering by bodily fluids restores the natural pH of the tissue to a value in the range of about 7.35 to about 7.45 within a time range of about 1 minute to about 8 hours, or in a time range of about 1 hour to about 6 hours.

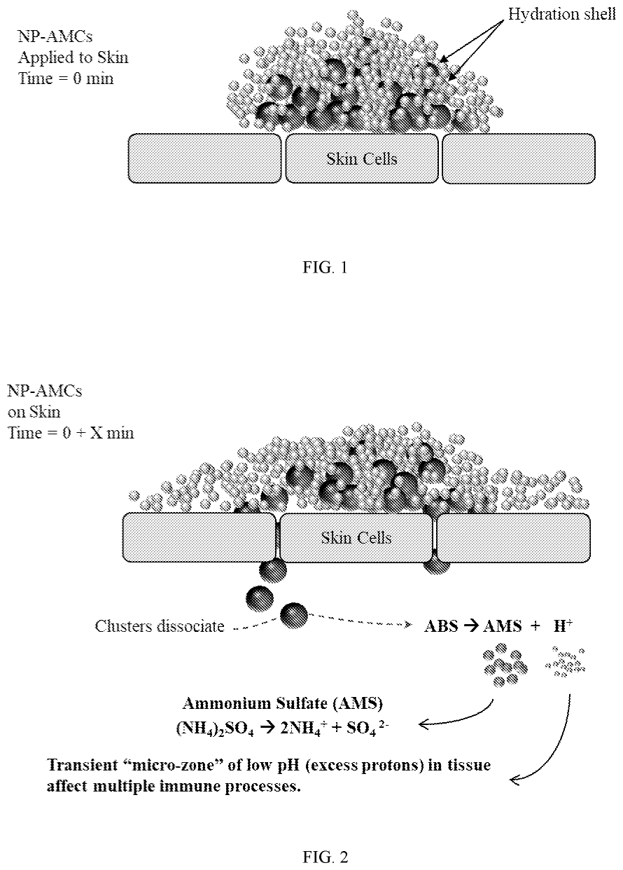

When compositions including NP-AMCs are applied to skin (FIG. 1), the NP-AMCs permeate into and through the skin and the wound bed into underlying tissue (FIG. 2). Acidification of a treated wound occurs as a result of NP-AMCs slowly destabilizing and dissociating due to the physicochemical parameters of the wound and surrounding tissue (e.g., pH, presence of reactive molecules, etc.). The NP-AMCs dissociate into the ionic chemical species that make up their constituent molecules, and the ionic species are released into the surrounding tissue. Water molecules present in the NP-AMCs become part of the local aqueous phase. Acid molecules dissociate into hydronium ions (H₃O⁺) (also referred to herein as protons) and a conjugate base. Ammonium salts dissociate into ammonium ions (NH₄⁺) and an anion that is a conjugate base of the acid used to form the ammonium salt. As one example, when the NP-AMCs include ammonium sulfate, ammonium bisulfate, sulfuric acid, and water, once applied to skin, the NP-AMCs permeate through the skin into the underlying tissue and dissociate into ammonium ions (NH₄⁺), sulfate ions (SO₄²⁺) and protons (H₃O⁺), see FIG. 2.

Not intending to be bound by theory, it is believed that transient acidification of skin and underlying tissue using methods and compositions disclosed herein stimulates a local immune response. A decrease in the pH of tissue can have far-reaching effects on the immune response. Moreover, studies have shown that different acids will result in different effects on the immune response. For example, decreasing the pH of tissue with HCI results in increased production of tissue necrosis factor (TNF), whereas lactic acid causes a decrease in TNF production and a decrease in production of some interleukins.

Not intending to be bound by theory, methods disclosed herein can be used to stimulate a signaling pathway to downregulate the production of a proinflammatory immune factor in an area of skin having a wound or skin disorder. In some examples, the proinflammatory immune factor is a proinflammatory cytokine selected from the group consisting of IL-1β, IL-6, IL-8, and TNF-α. As one example, dissociation of the NP-AMCs in vivo results in a presence of excess protons that are detected by G protein-coupled receptors (“GPCR”). Those receptors modulate the inflammatory response, which is the body's natural response to infection, trauma, injury, or other physical damage that causes cellular damage and/or introduction of materials or substances foreign to the body. In response to detecting the excess protons, the GPCRs stimulate aspects of the body's immune response, including intracellular signaling pathways to downregulate production of proinflammatory immune factors, such as but not limited to IL-8 and related signal molecules. As used herein a “signal molecule” refers to any extracellular or intracellular molecule or chemical compound, substance, or material that promotes or affects a specific reaction or pathway in a cell. Non-limiting examples of signal molecules include, among others, hormones, neurotransmitters, cytokines, and interleukins.

In some examples, a composition including NP-AMCs is applied to a wound at intervals of one or more hours to one or more days. For example, the composition can be applied to a wound at intervals of about 1 to about 8 hours, optionally in the range of about 4 to about 6 hours. The intervals may continue until the wound is fully healed, or alternatively the intervals may differ at different times during wound healing. For example, the composition may be applied more often during the first two days after a trauma occurs and less often thereafter. For example treatment after the first two days might be only on alternating days. Not intending to be bound by theory, with consistent continuous application of the NP-AMCs, the GPCRs may become desensitized to the presence of increased concentrations of protons in the environs of the wound. Varying the frequency of treatment can prevent such desensitization. In one example, intermittent treatment occurs on days 1 and 2 after the trauma, with no treatment on days 3 and 4, then resuming treatment on days 5 and 6. Optionally, this treatment schedule can be repeated until the wound healing has progressed to an obvious improved condition so that additional treatment is unnecessary.

In some examples, applying a composition including NP-AMCs to a wound increases oxygenation of the wound by providing an environment in which the local pH is more acidic, which allows oxygen to dissociate from hemoglobin more easily in vivo.

Dissociation of the molecular constituents of NP-AMCs provides benefits in addition to reducing the pH of the wound and surrounding tissues. One such benefit is that after dissociation, ammonium and sulfate ions are present in relatively high concentrations and can react in vivo to form ammonium sulfate [(NH₄)₂SO₄], which has analgesic properties. Thus, in some examples, a composition including NP-AMCs-is applied to an area of the skin including a wound as a means of increasing the rate of healing and provide analgesic relief from pain associated with the injury by releasing molecules of ammonium sulfate into the damaged skin layers and underlying tissue. In some examples, the analgesic property of a formulation containing NP-AMCs can be targeted to a specific location to relieve pain.

Described herein are methods for inhibiting the growth of an infectious microorganism in an area of skin having a wound or a skin disorder. The method comprises applying to the area of skin a composition including NP-AMCs and allowing the NP-AMCs to penetrate the skin and underlying tissues to decrease the pH of the skin and underlying tissues encompassing the wound or skin disorder. Decreasing the pH of a wound will inhibit growth of harmful microorganisms by rendering the wound microbiome unsuitable for growth of the microorganisms. As used herein the term “microbiome” refers to the collection of all microorganisms, including viruses, that live on or in a defined environment, such as but not limited to the body or a defined part of a body of a human or other mammal. The microbiome of a wound differs from a microbiome of healthy skin, with the wound microbiome being more suitable for the growth and proliferation of certain cell-damaging microorganisms. Decreasing the pH of the wound microbiome will make the wound microbiome unsuitable for cell-damaging microorganisms, and thus can prevent infection by those microorganisms.

In some examples of the methods disclosed herein, a composition including NP-AMCs is applied to the surface of a wound or infection site during the early stages of healing to maximize the benefit of the multiple modes-of-action of the molecular clusters. As explained herein, NP-AMCs can be used to derive one or more benefits including: (1) transient acidification of the wound bed of a trauma site or the site of an infection to prevent or slow growth of an infectious microorganism; (2) providing a source of protons that can be detected by GPCRs which will subsequently stimulate intracellular signaling pathways and diverse cellular responses that increase the rate of healing of the wound or infection site; (3) creating a localized transient low pH environment to ensure any ammonia produced by an infectious microorganism is maintained as ammonium ions as a means of minimizing or preventing lysis of erythrocytes in the wound or infected site; (4) directly affecting cell signaling in a manner that downregulates production of proinflammatory cytokines such as IL-1β, IL-6, and TNF-α, among others; and (5) increasing oxygenation of a wound by providing an environment in which the localized pH is more acidic which allows oxygen to more easily dissociate from hemoglobin in vivo.

Transient acidification of skin and the underlying tissue is controlled by the concentration of NP-AMCs in a pharmacologically acceptable formulation as well as the frequency of application of the formulation. Thus, this invention is an improvement over other methods to acidify a wound because NP-AMCs induce transient acidification of the skin and underlying tissue to lower the pH of the wound bed as well as underlying and adjacent tissue which provides benefits as detailed herein. The decrease in pH can be controlled to a desired pH value or range that favors aspects of the healing process, e.g., stimulation of angiogenesis, prevention of non-specific cytotoxicity caused by the enzymatic activities of matrix metalloproteinases produced by the body and infectious microorganisms.

The following examples are included to demonstrate preferred embodiments. It should be appreciated by those of skill in the art that the techniques disclosed in the examples which follow represent techniques discovered by the inventor to function well in the practice of the disclosed embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosed embodiments.

### EXAMPLES

### Example 1: Assessing Cytotoxicity of NP-AMCs

To assess if NP-AMCs were cytotoxic, two methods for evaluating cytotoxicity were employed. Cell viability was assessed using lactate dehydrogenase and ATP assays that measure different cellular responses to chemical or physical stimuli or stressors.

### Preparing the Cell-Lines

A commercially available canine progenitor epidermal keratinocyte cell line (CPEK; CELLnTEC, Zen-Bio Inc., Research Triangle Park, NC, USA) was used. Keratinocytes were cultured in 75 cm² flasks (Denville, Metuchen, NJ, USA) in CnT-09® media with serum (Canine Epithelial Proliferation Medium, CELLnTEC) at 37° C. with 5% CO₂ until confluence. Cells were then trypsinized using 5 mL of TrypLE (Life Technologies, Grand Island, NY, USA) for 5 min at 37° C. Then, approximately 1,500 cells were plated in a 24-well cell culture plate (Costar-Corning, Tewksbury, MA, USA) for 24 hours with CnT-09® media. The next day, once the cells were 80% confluent, they were exposed to CnT-09® media without serum for 24 additional hours (e.g., a starvation period) to remove the effects of the serum on the cells. The next day, the cells were exposed to four different concentrations of NP-AMCs (e.g., 1:10,000; 1:50,000; 1:100,000; and 1:500,000 v/v basis) in CnT-09® starving media with or without house dust mites (HDM) at a concentration of 1 mg/mL (Santoro et al., 2015. Ref: Santoro, D., et al. 2015. Evaluation of antimicrobial peptides and cytokine production in primary keratinocyte cell culture from healthy and atopic beagles. Exp. Dermatol. 24, 317-319. The HDM was selected as broad-spectrum inflammatory stimulus (Santoro et al., 2015). Wells without any NP-AMCs and HDM were used as negative controls (media only). Experiments were set up in duplicate and repeated five times independently.

### Cell Viability Assay I—Lactate Dehydrogenase (LDH) Assay

For the first viability assay, the Pierce™ LDH Cytotoxicity Assay (Thermo Fisher Scientific, Waltham, MA, USA) was used following the manufacturer's instructions. This assay provides a reliable colorimetric method for quantifying cellular cytotoxicity assays.

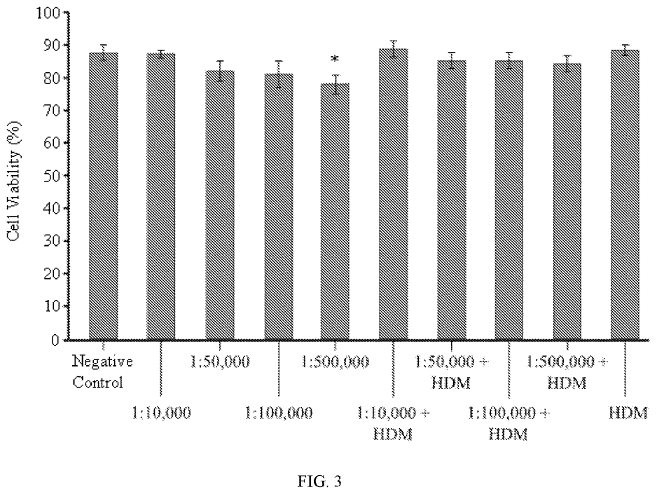

FIG. 3 is a bar graph of the average CPEK cell viability percent (LDH assay). The negative control is media only and represents an initial value. Values are shown for 24 hour exposure to NP-AMCs at different concentrations, exposure to house dust mites (HDM), and exposure to a combination of NP-AMCs and HDM. *: p<0.05=significantly different from baseline (media). Bars: standard error of the mean. As illustrated in FIG. 3, the LDH cell viability assay showed no statistically significant difference between any of the conditions tested and the baseline (media only) except for the NP-AMCs used at a 1:500,000 dilution. However, the reduction in cell viability in the 1:500,000 dilution of NP-AMCs, e.g., 10%, was within reasonable experimental error. Considering the results for all concentrations of NP-AMCs evaluated, overall, there was no obvious cytotoxicity caused by NP-AMCs.

### Cell Viability Assay II—CellTiter-Glo® 2.0 Assay

For the second cell viability assay, the CellTiter-Glo® 2.0 Assay (Promega, Madison, WI, USA) was used following the manufacturer's instructions. This assay measures the amount of ATP present in the cells before and after exposure to a chemical agent or other stressor. The mean luminescence value of each of the five-tested concentrations and the positive control (e.g., kit reagent) were compared to the average of the negative control to determine the effect of the treatments on cell viability.

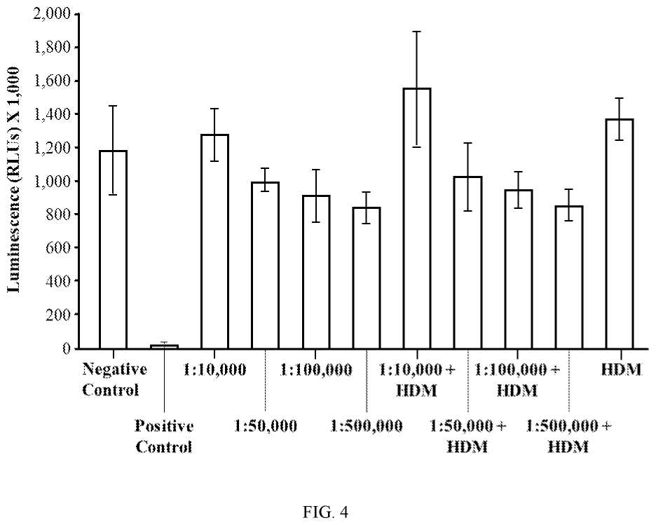

FIG. 4 is a bar graph of the average CPEK cell viability as a function of luminescence (CellTiter-Glo® 2.0 assay). The negative control is media only and represents an initial value. Values are shown for 24-hour exposure to NP-AMCs molecular clusters at different concentrations, exposure to HDM, or exposure to a combination of NP-AMCs and HDM for 24 hours. RLU: relative luminescence unit. Bars: standard error of the mean. As illustrated in FIG. 4, the CellTiter-Glo® 2.0 Assay results demonstrated that there were no statistically significant differences between the different treatments with NP-AMCs and/or HDM versus the culture medium baseline. This illustrates the lack of cytotoxicity when cells were treated with concentrations of NP-AMCs that are in the range of the amount in a formulation for treating a skin or tissue malady.

### Example 2: Assessing if NP-AMCs Have a Measurable Effect on Production of Selected Cytokines and Interleukins

The Milliplex MAP canine Multiplex® assay (Millipore, Billerica, MA, USA) and the TGF-β1 quantakine assay (R & D systems, Minneapolis, MN, USA) were carried out according to manufacturers' protocols. The assays used allowed for measuring six cytokines, Granulocyte-Monocyte-Colony stimulating factor [GM-CSF], Interleukin [IL]-8, IL-10, IL-18, keratinocyte-derived chemokine [KC/CXCL1], and monocyte chemotactic protein-1 [MCP-1/CCL2].

Data analysis was performed using the MasterPlex QT 1.0 system (MiraiBio, Alameda, CA). A five-parameter regression formula was used to calculate the sample concentrations from the standard curves.

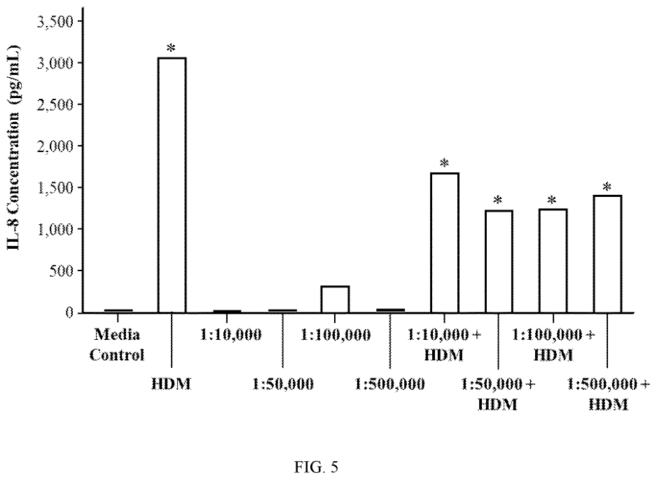

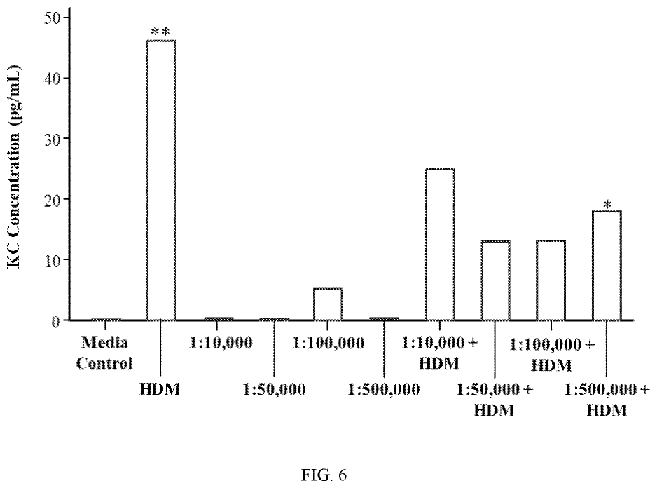

FIGS. 5 and 6 show that two of the six selected cytokines, e.g., IL-8 and KC-like (also known as CXCL-1), were detected in cells treated with NP-AMCs. The other cytokines, GM-CSF, IL-10, IL-18, MCP-1/CCL2, and TGF-β, were below detection limit. As expected, when compared to the baseline (i.e., culture medium only), significant increases in IL-8 and KC cytokines were measured after exposure to HDM (p=0.001 and p=0.002, respectively). There was no difference between baseline and IL-8 and KC after exposure to any of the concentrations of NP-AMCs evaluated (p>0.05). However, cells treated with combinations of HDM and NP-AMCs produced significantly more (p<0.05) IL-8 (compared with baseline). FIG. 5 is a bar graph of the median CPEK inflammatory cytokine production (Interleukin-8 [IL-8]) before (media control) and after exposure to (a) NP-AMCS molecular clusters at the indicated concentrations, (b) house dust mites (HDM), and (c) combination of both for 24 hours. IL-8 minimum detection concentration=21.7 pg/mL). *: p<0.05=significantly different from baseline (media control). The amounts of IL-8 produced by all concentrations of NP-AMCs were less than in HDM plus NP-AMCs. Treatment of the cells with selected concentrations of NP-AMCs did not result in any significant differences in the amounts of IL-8 produced. However, when the cells were exposed to combinations of NP-AMCs and HDM, there were reductions of between 59% and 65% in IL-8 secretion versus HDM alone.

The results of NP-AMCs alone or plus HDM on KC production (FIG. 6) had an overall pattern similar to that for IL-8. In this case, a significant increase in production of KC, compared with the baseline, was only detected in the lowest concentration of NP-AMCs (e.g., the 1:500,000 dilution) (p=0.04).

The above results are partially in agreement with previous studies in which only a few pro-inflammatory cytokines were detected in CPEK lines (see Kimura et al., 2012. Production of GM-CSF mediated by cysteine protease of Der f in canine keratinocytes. J. Vet. Med. Sci. 74, 1033-1036 and White et al., 2014. Canine progenitor epidermal keratinocytes express various inflammatory markers, including interleukin-8 and CD40, which are affected by certain antibiotics. Vet. Dermatol. 25, 493-502, e81-2).

Results indicate that cells treated with low concentrations of NP-AMCs secrete less of the pro-inflammatory mediators after stimulation, facilitating the reestablishment of normal tissue and cellular homeostasis. The cytokines, IL-8 and KC, which had increased production following treatment with NP-AMCs (with or without HDM) play a significant role in chemotaxis of inflammatory cells, in particular neutrophils. (see Gillitzer and Goebeler, 2001. J. Leukoc. Biol. 69, 513-521 and Balaji et al., 2015. Adv. Wound Care [New Rochelle] 4, 660-672.

### Example 3: Effects of NP-AMC Containing Solution on Pig Skin

Samples of pig skin were dosed with 100 μg DermaBase Cream amended with 2.5% NP-AMCs solution and a 1:10,000 dilution of the NP-AMCs stock solution which contained 2.38% (w/w) NP-AMC. Pig skin samples were dissected from a fresh pork shoulder roast purchased from a local supermarket.

### Paper Towel Substratum Saturated with an Anthocyanin Solution (pH: 8.5)

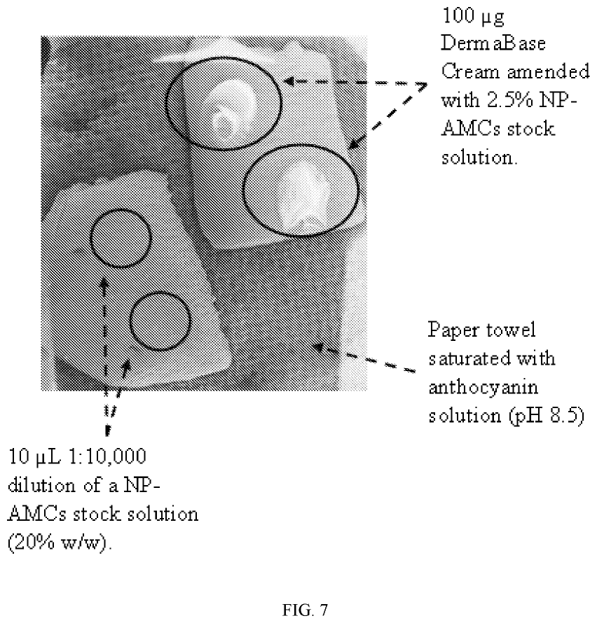

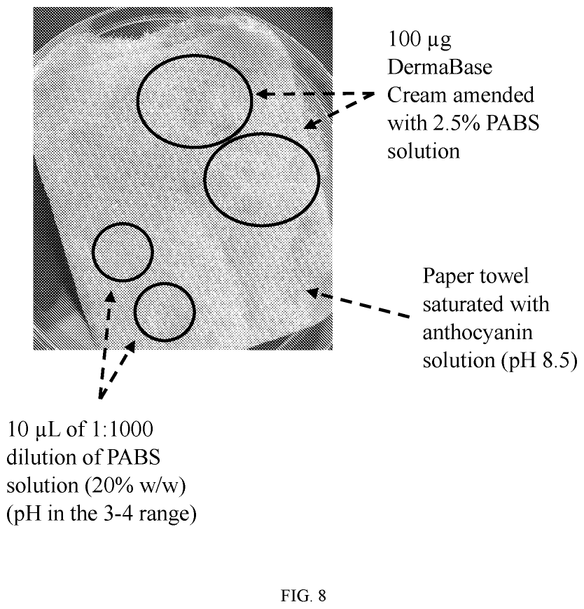

The dermal layers with a minimum of the underlying adipose tissue were aseptically collected and placed in sterile plastic Petri dishes onto a paper towel substratum saturated with an anthocyanin solution (pH 8.5). After blotting the surfaces with paper towels and allowing the skin samples to air-dry for 30 minutes, 100 μg DermaBase Cream amended with 2.5% NP-AMC stock solution were placed onto two locations of a first sample and 10 μL of a 1:10,000 dilution of a NP-AMC stock solution were placed onto two locations of a second sample. The locations and concentrations of the NP-AMC solutions are shown in FIG. 7.

After incubating for 6 hours at room temperature (ca. 72° F.), the skin samples were removed, and the anthocyanin-saturated paper towel was photographed (FIG. 8). There was a change in the anthocyanin pH indicator (e.g., from a light green color, indicating a pH in the 8-9 range, to a light pink color, indicating a pH in the 3-4 range). Not intending to be bound by theory, these results show that the acidic clusters permeated the dermal layers and caused a change in pH below the skin.

### Whatman #3 Filter Paper Saturated with an Anthocyanin pH Indicator Solution (pH: 12)

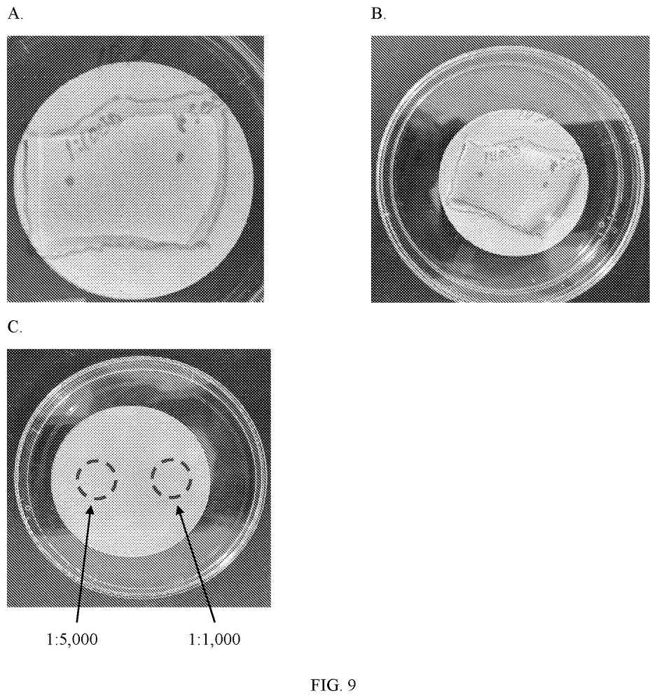

Two layers of Whatman #3 filter paper saturated with the anthocyanin pH indicator solution with the pH adjusted to 12 by adding 0.25% (w/v) NaOH. 10 μL of the NP-AMC solution diluted 1:1000 and 1:5000 was placed onto the surface of a pig skin sample the filter paper in a sterile Petri dish. After incubating at room temperature for 4 hr, photographs were taken. The photograph in FIG. 9 panel A was taken at time=0 (immediately after placing the 10 μL samples at the locations indicated by the dots. The photograph in FIG. 9 panel B was taken after 4 hr incubation. The photograph in FIG. 9 panel C was also taken after 4 hr incubation and is a bottom view showing change in color in the area where samples were placed onto the skin samples. For the bottom view, the Petri dish with lid in place was carefully flipped 180 degrees. The outline of the skin sample is apparent where the pH decreased to ~4 (pink color) as a result of NP-AMC permeating the dermal layers.

### Example 4: Effects of NP-AMC Containing Solution on Fresh Ham Hock

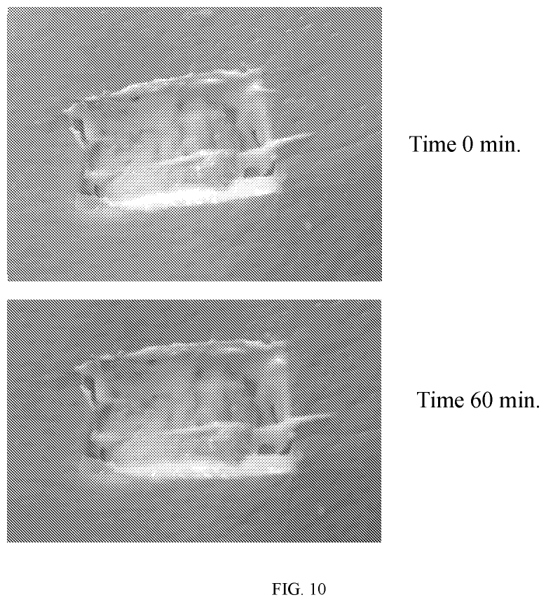

Fresh ham hock was obtained from a local supermarket and allowed to warm to room temperature (e.g., 72° F). Full thickness wounds were made by excision of the dermal layers with a scalpel fitted with a #10 blade. As illustrated in FIG. 10, the full thickness wound was about 1 cm by about 1.5 cm. The wound was treated with 100 μg of DermaBase Cream amended with 2.5% (w/w) of a 20% (w/w) PABS solution. After treatment, the wound was incubated at room temperature for 60 min and photographed (FIG. 10). The appearance of the wound did not change during the incubation period. Close visual examination of the wound and treatment indicated there was no detectable change in appearance. This is consistent with other observations of wounds in porcine skin, see Example 3.

### Example 5: Comparison of Fresh Ham Hock Skin Treatment Subjected to Different Treatment Conditions

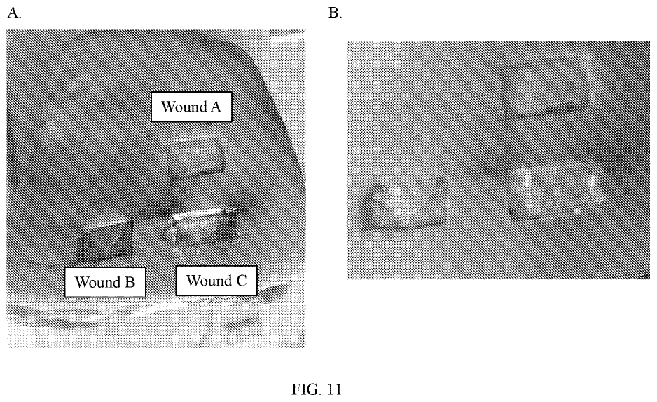

In this example, a fresh ham hock was obtained from a local supermarket and allowed to warm to room temperature (e.g., 72° F). After drying the surface with paper towels, three full thickness wounds were made by excision with a scalpel fitted with a #10 blade. As illustrated in FIG. 11, the dimensions of the full thickness wounds were about 1 cm by about 1.5 cm. Wound “A” was an untreated control, Wound “B” was filled with 100 uL of the anthocyanin pH indicator solution, and wound “C” was filled with 100 μg of DermaBase Cream amended with 2.5% (w/w) of a 20% (w/w) NP-AMCs solution. After wound “C” was treated and allowed to incubate at room temperature for 30 min, 100 uL of the anthocyanin pH indicator solution was added. The appearance of wounds “A” and “B” did not change during the incubation period. However, in wound “C”, the pH indicator changed color (e.g., from blue [indicating a pH in the range of 7 to 8] to pink [indicating a decrease in pH to a value in the range of about 3 to about 4]). Close visual examination of the surface of underlying tissue indicated the pH was also in the range of about 3 to about 4.

### Example 6: Acidification Caused by NP-AMCs on Pig Skin Samples

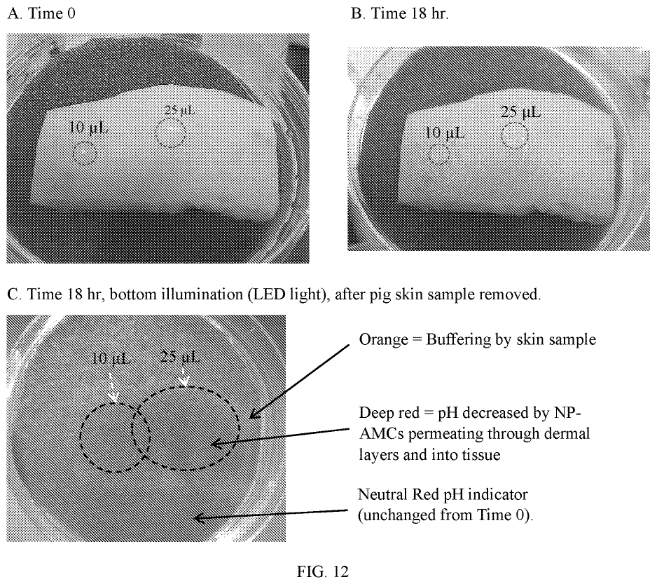

A pig skin sample (dermal layers with ca. 3-5 mm of attached adipose tissue) was placed onto a double layer of a paper towel saturated with a solution of 1% (w/v) Neutral Red in distilled water in a sterile Petri dish. The pH indicator solution was used as prepared. Two volumes (e.g., 10 and 20 μL) of a 10% NP-AMCs solution (10% w/v as NP-AMCs in distilled water) were placed onto the pig skin sample in areas that were dried via blotting with a paper towel. The treated pig skin sample was incubated overnight at room temperature (~73° F). After the incubation period, the pig skin sample was photographed and then removed so that the Neutral Red-soaked paper towel could be examined and photographed (FIG. 12).

As illustrated in FIG. 12, panels A-C, the initial pH of the Neutral Red solution was in the acidic range (e.g., below pH 6.8). The samples of the NP-AMCs solution were visible (FIG. 12, panel A). After the 18-hr. incubation, the 10 and 20 μL NP-AMCs solutions had seemingly permeated the dermal layers of the skin sample (FIG. 12, panel B). Thereafter, the skin sample was removed and the Petri dish with the absorbent paper layer was placed over a LED light and photographed (FIG. 12, panel C). The outline of the skin sample (area with an orange color) was visible. The color change of the Neutral Red pH indicator from red to orange was caused by the pH of the skin sample. The color change of the pH indicator in the areas beneath the locations where the 10 and 20 μL samples of the NP-AMCs solution had been applied were also visible. Not intending to be bound by theory, the deep red color indicates a pH less than 6.8, the result of acidification caused by the NP-AMCs.

### Example 7: NP-AMC Containing Cream to Treat Bite Wound on Human Hand

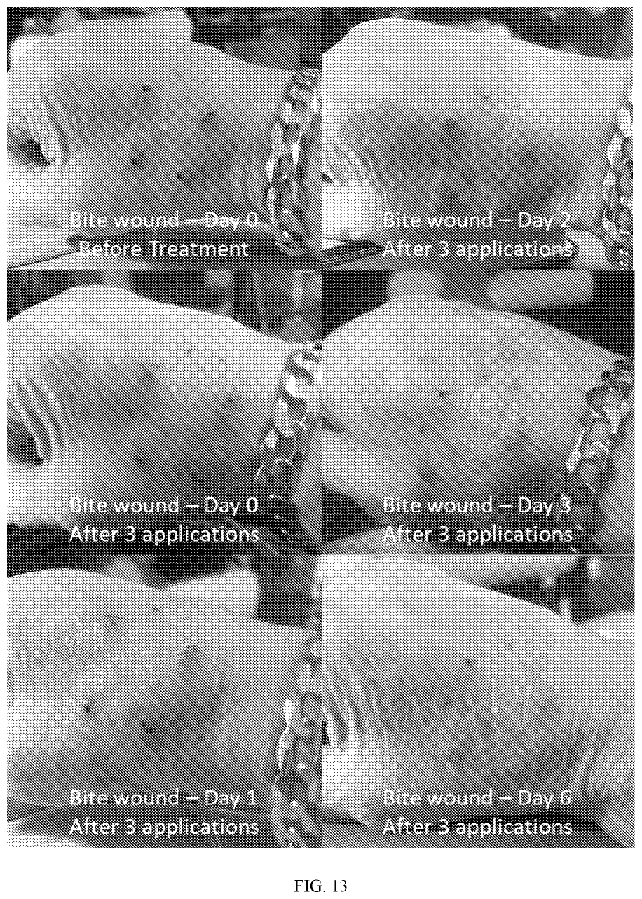

A Caucasian male volunteer, age 69, suffered a cat bite wound on his hand. FIG. 13 shows the wound treatment progression after six days. The cream was applied three times a day at 4 to 6 hr intervals (10 a.m., 2 p.m., and 8 p.m.). Images of the wound were taken 2 hrs after final application and 12 hrs after initial application (~10 p.m.). Wound was treated daily for six consecutive days and data was recorded for days 0, 1, 2, 3, and 6 days post-treatment. Not intending to be bound by theory, close visual examination of the surface of underlying tissue shows significant improvement on wound closure after six days of treatment where three of the four puncture wounds are completely closed, and the fourth puncture wound is substantially closed. The subject had previously suffered similar cat bite wounds, and he explained that similar wounds typically take many weeks to a few months to heal to the same condition achieved in six days using the NP-AMC.

It should be apparent to those skilled in the art that the foregoing is merely illustrative and not limiting, having been presented by way of example only. Numerous modifications and other embodiments are within the scope of one of ordinary skill in the art and are contemplated as falling within the scope of the invention. Although many of the examples presented herein involve specific combinations of method acts or system elements, it should be understood that those acts and those elements may be combined in other ways to accomplish the same objectives.

1. A method of treating a damaged area of skin having a wound or a skin disorder, the method comprising applying to the damaged area of skin a composition comprising a pharmaceutically acceptable carrier and a plurality of nanoparticles, each nanoparticle comprising a self-assembled acidic molecular complex comprising an ammonium salt and an acid (“NP-AMC”), wherein the composition is applied in an amount effective to reduce a pH of the damaged area of skin and produce a treated area of skin.

2. The method of claim 1, wherein the pH of the treated area of the skin is from pH 2 to pH 7.

3. The method of claim 1, wherein the pH of the treated area of skin is lower than the pH of the damaged area of skin by 1 pH unit.

4. The method of claim 1, wherein the pH of the treated area of skin remains lower than the pH of the damaged area of skin for a time period ranging from about 5 minutes to about 6 hours.

5. The method of claim 1, wherein the wound or skin disorder comprises an infectious microorganism, and wherein the composition is applied in an amount effective to inhibit growth of the infectious microorganism.

6. The method of claim 1, wherein applying the composition comprises topically applying the composition to the damaged area of skin and to skin and/or tissue surrounding the damaged area of skin.

7. The method of claim 1, wherein the composition comprises a pH ranging from 1 to 6.

8. The method of claim 1, wherein the concentration of the nanoparticles in the composition is from about 0.005% to about 50% by weight.

9. The method of claim 1, wherein the nanoparticles have an average diameter ranging from about 5 nm to about 2000 nm.

10. The method of claim 1, wherein the damaged area of skin comprises a trauma site or a site of infection.

11. The method of claim 1, wherein the wound is selected from the group consisting of a scrape, a scratch, a cut, an abrasion, a puncture, an animal or insect bite or sting, and a reaction to contact with a chemical that damages healthy skin.

12. The method of claim 1, wherein the wound or skin disorder is an acute wound or skin disorder.

13. The method of claim 1, wherein the wound or skin disorder is a chronic wound or skin disorder.

14. The method of claim 1, wherein the ammonium salt is selected from the group consisting of ammonium chloride (NH₄Cl), ammonium sulfate ((NH₄)₂SO₄), ammonium bisulfate (NH₄HSO₄), ammonium carbonate ((NH₄)₂CO₃), ammonium bicarbonate (NH₄HCO₃), ammonium iron(II) sulfate ((NH₄)₂Fe(SO₄)₂.6H₂O), ammonium formate (NH₄HCO₂), ammonium acetate (C₂H₇NO₂), ammonium borate (H₁₂BN₃O₃), ammonium nitrate (NH₄NO₃), and ammonium phosphate ((NH₄)₃PO₄), and wherein the acid is selected from the group consisting of acetic acid, boric acid, carbonic acid, hydrochloric acid, nitric acid, phosphoric acid, sulfamic acid, sulfuric acid, and formic acid.

15. The method of claim 1, wherein the ammonium salt is selected from the group consisting of ammonium chloride (NH₄Cl), ammonium carbonate ((NH₄)₂CO₃), ammonium bicarbonate (NH₄HCO₃), ammonium iron(II) sulfate ((NH₄)₂Fe(SO₄)₂.6H₂O), ammonium formate (NH₄HCO₂), ammonium acetate (C₂H₇NO₂), ammonium borate (H₁₂BN₃O₃), ammonium nitrate (NH₄NO₃), and ammonium phosphate ((NH₄)₃PO₄), and wherein the acid is selected from the group consisting of acetic acid, boric acid, carbonic acid, hydrochloric acid, nitric acid, phosphoric acid, sulfamic acid, and formic acid.

16. The method of claim 1, wherein the ammonium salt is ammonium sulfate and/or ammonium bisulfate, wherein the acid is sulfuric acid, and wherein the NP-AMCs further comprise water.

17. The method of claim 1, wherein the treating comprises reducing inflammation.

18. The method of claim 1, wherein the composition is applied in an amount effective to stimulate the signaling pathway to downregulate the production of a proinflammatory immune factor.

19. The method of claim 18, wherein the proinflammatory immune factor is a proinflammatory cytokine selected from the group consisting of IL-1β, IL-6, IL-8, and TNF-α.

20. The method of claim 1, wherein the composition is in the form of a solution, a suspension, an emulsion, a powder, a gel, an ointment, a cream, or a paste.
